(19) United States
(12) Patent Application Publication
(10) Pub. No.: US 2025/0000703 A1
(43) Pub. Date: Jan. 2, 2025

(54) OPHTHALMIC MASK, OPHTHALMIC DEVICE AND USE THEREOF

(71) Applicant: A.R.C. Laser GmbH, Nürnberg (DE)

(72) Inventors: Reinhardt THYZEL, Eckental (DE); Colin Francis THYZEL, Hallstädterweg (DE)

(21) Appl. No.: 18/711,443

(22) PCT Filed: Nov. 18, 2022
(86) PCT No.: PCT/EP2022/082444

§ 371 Date: May 17, 2024

(30) Foreign Application Priority Data
Nov. 18, 2021 (DE) 102021130135.1

(51) Int. Cl. A61F 9/009; A61F 9/008

(52) U.S. Cl. CPC A61F 9/009; A61F 9/00821; A61F 2009/00868; A61F 2009/00891

(57) ABSTRACT

An ophthalmic device comprises an ophthalmic mask having a first side for application or support on the eyeball, and a second side facing away from the first side, a plurality of positioning markers for positioning a light guide on the positioning marker, the mask body being opaque in a planar central area for a light used to treat the eye, an area size of the central area being adapted to an area size of the pupil and iris of an eye to be treated. The positioning markers are formed on the mask body as indentations on the face-sided edge extending from the second side into the mask body. The mask body is transparent to the light in the area of the indentations. A dimension of the indentations is adapted to a dimension of a light guide provided for the punctual application of light.

The invention relates to an ophthalmic mask and an ophthalmic device.

The ophthalmic mask or device is intended in particular for use in the treatment or therapy of the eye, for example glaucoma, and in particular for use in combination with a light applicator, in particular a laser applicator, for the treatment or therapy of the eye.

Glaucoma is a serious eye disease that can lead to partial or complete blindness due to damage to the retina. A common cause of glaucoma is increased intraocular pressure. Increased intraocular pressure can occur, for example, if the flow channel for the intraocular fluid in the area of the ciliary muscles and the trabecular meshwork is narrowed or if there is too much intraocular fluid without appropriate pressure equalization.

Eye treatment devices are now known in which the flow channel is widened again and/or the ciliary muscle is partially destroyed by means of laser light shone through the cornea, thereby improving the outflow of ocular fluid, for example so-called cyclophotocoagulation using continuous high-intensity laser light from the infrared spectrum.

EP 3 160 379 B1 discloses a treatment probe or applicator for treating a portion of the eye comprising an elongate body defining a handle having a proximal end and a distal end, and a solid contact element connected to the distal end of the elongate body. The contact element includes a convex contact surface for contact with the surface of the eye. Further, the probe comprises a treatment fiber that extends longitudinally in the elongated body and from the distal end of which laser light emerges for treatment. The distal end of the treatment fiber terminates at the convex contact surface and is supported laterally all around by the surrounding solid material of the contact element. This treatment probe is used to treat glaucomas in particular.

WO 2018/152020 A1 describes a laser-based eye treatment device with a laser light source for therapeutic laser light and an eye mask for positioning on the eye. In the eye treatment device of WO 2018/152020 A1, the mask body is irradiated during a glaucoma treatment by the laser light source positioned at a distance from the mask, the mask body acting like an irradiation grid and having a plurality of openings. During treatment, the mask body is irradiated and the light incident on the mask body outside the openings is reflected. Only the light that hits the openings can pass through the mask and has a therapeutic effect in the eye.

The invention is based on the objective of providing an alternative ophthalmic mask and an alternative ophthalmic device, in particular for use in a treatment or therapy of the eye with light, in particular laser light. The alternative ophthalmic mask and the alternative ophthalmic device are intended to be applicable in particular for the treatment of glaucoma.

This objective is solved according to the invention with the features of the independent patent claims. Further embodiments according to the invention result from in the dependent patent claims.

According to one embodiment, an ophthalmic mask is provided for application to an eyeball of an (open) eye. The mask is designed to be placed on the eye when the eye is irradiated with light, in particular laser light, by a light guide, e.g. with a light applicator comprising an optical fiber or a light guide which is designed to irradiate light into the eye, e.g. via the cornea of the eye. The light may be laser light in particular. The ophthalmic mask is particularly suitable for use in the treatment or therapy of glaucoma. The mask makes it possible to cover the eye, in particular in the area of the pupil and/or pupil opening, in order to prevent light or laser light that is harmful to the retina of the eye, for example, from entering the eye and hitting the retina, while allowing irradiation of certain areas of the inside of the eye.

The mask includes:
a) a shell-like mask body with a first side for application or support on the eyeball, and a second side facing away from the first side,
b) a plurality of positioning markers for positioning a light guide in or at the positioning marker, wherein the positioning markers are visible at least on the second side;
c) wherein at least in a two-dimensional, in particular two-dimensionally extended, central area the shell-like mask body is opaque for a light, in particular laser light, used to treat the eye,
d) an area size of the central area is adapted to an area size of the pupil and the iris of an eye to be treated, and
e) the positioning markers are arranged outside the central area,
whereby
f) the positioning markers on the mask body are formed as indentations on the face-sided edge, for example U-shaped or ring-segment-shaped indentations, and/or as recesses extending from the second side into and/or through the mask body, and the mask body is transparent to the light in the region of the indentations and/or recesses, and in particular is opaque outside the indentations, and wherein
g) a dimension of the indentations and/or recesses is adapted to a dimension of a light guide provided for the punctual application of light, in particular laser light, or a dimension of the indentations and/or recesses is designed in terms of shape and/or size such that the light guide, in particular a distal end designed to emit the light, may be positioned thereon and/or therein, in particular such that the light may be irradiated into a treatment area in the eye associated with the indentation or recess or defined by the latter.

The mask therefore makes it possible to irradiate the eye with light in defined areas or treatment areas, whereby the areas or treatment areas are predefined by the recesses and/or indentations after being placed on the eye.

According to one embodiment, the mask comprises a plurality of face sided indentations, wherein face sided indentations of at least one group of face sided indentations are distributed in the circumferential direction of the mask body, in particular distributed substantially equidistantly.

The face side is understood to be, in particular, a laterally outwardly pointing or laterally outer edge or side of the mask body. The mask body may have a curvature, in particular such that the contact surface or side intended for contact with the eye, in particular the cornea, is essentially or at least to a certain extent curved, corresponding to the curvature of the cornea of the human or animal eye.

According to one embodiment, the mask may comprise a plurality of the recesses, wherein at least one group of recesses is arranged along a path extending at least in sections in a circular or elliptical shape on the mask body, in particular substantially equidistantly. The shape of the path may, for example, have the aforementioned shape in plan view of the mask, whereby the plan view is, for example, related to the mask positioned on the eye. Circular or elliptical paths in sections should also be understood to mean, for example, spiraled paths at least in sections.

According to one embodiment, at least one indentation in the cross-section may be at least partially in the form of a circular segment and/or at least one recess in the cross-section may be at least partially in the form of a circular surface, whereby a radius assigned to the circular segment or the circular surface may be adapted to a radius of the light guide provided for the punctual application of light. For example, the circular segment may be a U-shaped or C-shaped indentation on the edge that extends inwards into the mask body. In a central area, for example, the recess may have a shape that is adapted to a circular area or circular shape.

According to one embodiment, at least one indentation and/or at least one recess may have an elongated shape in a circumferential direction around the central area or in a radial direction to the central area. For example, the shape of the indentation or recess may be adapted to the shape of an elongated hole, whereby inner longitudinal ends of the elongated hole shape may be rounded.

According to one embodiment, at least one indentation and/or at least one recess may be formed in a circumferential direction around the central area in the form of a circular ring segment or a circular ring. In this case, and for example also in the case of an elongated hole shape, the light guide may be inserted into the recess/indentation or positioned therein and displaced along the circular ring or elongated hole. Accordingly, extended treatment areas may be defined by such forms of the indentation/recess.

According to one embodiment, the indentations and/or recesses may be arranged in the manner of a cobweb pattern on the mask body. A visual marking may be applied to the cobweb pattern, for example on the side facing away from the eye, for example as a guide for the user to guide the light guide during successive positioning of the light guide in the indentations/recesses.

According to one embodiment, at least one, preferably several, groups of recesses may be provided, wherein center points of at least one, preferably each, group of recesses may be arranged on a respective path, in particular a circular path, running around the central area, and wherein a smallest diameter (D) of the path, in particular a diameter of a circular path, is preferably in the range from 9.5 mm to 13 mm, in particular from 10 mm to 12 mm.

According to one embodiment, the mask comprises a groove structure provided on the second side of the shell body, which is preferably opaque to the light outside the recesses/indentations, wherein in each case two recesses or a recess and an indentation are connected by a groove extending therebetween, wherein the groove is set up as a guide aid for the light guide. For example, the groove structure or a groove may be provided to guide the distal end of the light guide when it is repositioned from one indentation/recess to the next indentation/recess.

According to one embodiment, a side wall of one or more indentations and/or recesses may have, at least in sections, a conical taper from the second side towards the first side. A minimum diameter of a recess or indentation that, at least in sections, tapers conically may essentially correspond to the diameter of the light guide. A convergence angle of the conical taper may be larger on a side of the recess facing the central area than on a side of the recess facing away from the central area. In particular, a side wall is understood to be a wall that runs essentially perpendicular or at an angle to the surface of the mask body. The conical shape may make it easier for the user to insert the distal end of the light guide, i.e. the end at which the light guide emits light.

According to one embodiment, a side wall of one or more indentations and/or recesses may be at least partially cylindrical and, at least in sections, extend substantially perpendicular to the first side.

In one embodiment, the mask may have at least two layers, or multiple layers, or two shells. For example, the mask body may have at least one outer shell facing away from the eye after being placed on the eye and an inner shell facing the eye. Further layers or shells may be arranged between these two shells or layers. The inner shell or layer may be transparent to light. The outer shell may be opaque to light. The shells are designed in such a way that transparent passages are formed in the area of the recesses/indentations for the light. The inner shell or layer may be designed in the manner of a contact lens, for example, in such a way that adhesion and positioning on the eye is possible, in particular without injuring the eye or the cornea. The outer layer or shell may, for example, be designed to absorb or reflect light, for example in such a way that the light outside the indentations/recesses cannot penetrate the mask body.

According to one embodiment, the mask may further comprise a plurality of visual markings at the second side of the mask body, respectively assigned to the respective positioning markers and indicating a sequence of a punctual irradiation. The markings may be printed, embossed, punched or milled, for example. The markings may indicate to the user a processing sequence or a processing sequence for positioning the light guide in the recesses/indentations.

According to one embodiment, an ophthalmic device for the treatment, in particular laser treatment, of an eye is provided. The device comprises at least one ophthalmic mask formed to rest on an eyeball of the eye according to one of the embodiments according to the invention described herein, a light applicator, in particular a laser applicator, with a light guide which has a free distal end, the shape of which is adapted to the shape of the positioning markers and may be selectively positioned directly at or in the positioning markers, so that after positioning the distal end directly at or in one of the positioning markers, the eye may be selectively exposed locally to light, in particular laser light (L, L′, L″), through the distal end of the light guide.

According to one embodiment, the ophthalmic device further comprises a light source, in particular a laser light source, preferably an Nd:YAG laser source, further preferably a pulsed laser source, which is connected or connectable to the light applicator via a connection line, wherein light that may be generated by the light source may be coupled into the light guide for emission at the distal end via the connection line.

According to an embodiment, in particular one which may be claimed separately, an ophthalmic device is provided, in particular according to one of the embodiments described above, in particular an ophthalmic instrument.

The ophthalmic device is designed for the treatment, in particular laser treatment, of an eye, in particular in connection with the treatment of glaucoma.

The ophthalmic device, hereinafter also referred to as “device” for short, comprises a support body with a first side for direct (or: immediate) contact with an eyeball, in particular for direct (or: immediate) contact with the cornea of an eye. The support body comprises a grip element, for example a handle, e.g. a handle-like grip. The grip element is designed for holding the support body on the eyeball and/or for pressing it against the eyeball. In particular, it is provided that the support body may be placed and/or pressed directly onto the eye with the first side, in particular onto the cornea in the area of the pupil, and may be held in position by the grip element by a user, for example by an attending physician.

The device further comprises a guide body, in particular designed to guide a light guide held or fixed thereto, in particular a free end of a light guide, for example a laser light guide, held or fixed thereto.

The guide body comprises at least one retaining element or fixing element, for example in the form of a channel, whereby the channel may be open on both sides or in the form of a blind hole. The retaining element or fixing element is set up and designed for releasably holding a free end of a light guide on the guide body in such a way that, when the support body is placed (or: positioned) on the eyeball, light, e.g. laser light, in particular laser light pulses, may be applied, by the light guide, to the eyeball or components thereof in a targeted manner, in particular locally targeted manner.

The guide body is movably mounted on the support body, in particular in a sliding manner, in such a way that the retaining element or fixing element moves along a predetermined path when the guide body moves relative to the support body. If the free end of the laser light guide is held on or by the retaining element or fixing element, for example by the free end being held, e.g. inserted into the channel, the free end may be moved, via the movement of the guide body, along the path accordingly. Along the path, light, in particular laser light, may be irradiated continuously or locally selectively onto the eye or components thereof during operation of the device with the laser guide inserted. If, for example, the path is set up such that the path runs at least partially in the area of the ciliary muscle of the eye when the support body is in place, the ciliary muscle may be exposed to laser light, for example. As a result, the ciliary muscle contracts and thereby improves the outflow of intraocular fluid via the trabecular meshwork, thus reducing the intraocular pressure for the treatment of glaucoma.

According to one embodiment, the device may, for example, be designed in exactly two or exactly three parts. In a two-part design of the device with two, in particular separate, components, the support body with grip element on the one hand may form a first component and the guide body on the other hand may form a second component. In this case, the support body and the grip element may, for example, be formed in one piece and/or be connected to each other, for example by a material connection, in such a way that the support body and the grip element cannot be separated from each other in a non-destructive manner. In a three-part design, the device may consist of the three components i) grip element, ii) support body and iii) guide body. The grip element may, for example, be detachably attached to the support body, however, in particular, in such a way that the unit consisting of the grip element and the support body may be handled as a single unit.

In all embodiments, for example whether two-part or three-part, the guide body may be detachably, in particular non-destructively detachable, e.g. loss-proof, attached to or coupled with the support body. If the guide body is coupled to the support body, in particular a unit results that may be individually handled as such by the user during intended use, e.g. that may be handled with one hand, and which forms, in particular, an ophthalmic device or an ophthalmic instrument.

In embodiments, the device is preferably set up so that the path is essentially concentric to the eye axis or visual axis of the eye, or that the path is adapted to the structure of the eye and the position of its components, in particular the pupil or iris.

According to a particularly advantageous embodiment, the support body of the ophthalmic device has a ring bead on the first side for, in particular, direct (or: immediate) contact with the eyeball. The ring bead may, for example, be torus-shaped on the first side, e.g. protruding. The ring bead preferably has a convex curvature when viewed from the first side. Preferably, the ring bead is arranged in such a way that placing the support body on the eye, in particular the cornea, causes essentially no damage to the eye/cornea.

According to particularly advantageous embodiments of the ophthalmic device, at least one retaining element or fixing element, preferably all retaining elements or fixing elements, is/are designed as a channel. The channel may, for example, be designed as a channel open on both sides or in the form of a blind hole, which extends, for example, in the direction of the first side. In the case of a blind hole, the channel may be closed towards the first side by a base that is transparent to light. The channel is designed in particular in such a way, for example in diameter and/or length, that the free end of the light guide may be inserted into it. Furthermore, the channel is designed and arranged in such a way that light is applicable to the eyeball or components thereof through the inserted free end of the light guide, preferably at least locally along the predetermined path when the guide body is moved relative to the support body. An opening of the channel is preferably oriented in the direction of the second side facing away from the first side, so that the free end of the light guide may be inserted into the channel from the second side, with a light-emitting surface of the light guide facing the first side or the bottom of the channel.

According to advantageous embodiments of the ophthalmic device, the support body has a slide bearing surface and the guide body has a complementary slide bearing surface, which form a slide bearing, preferably an axial slide bearing, wherein the slide bearing is preferably designed as a tapered slide bearing. Such a slide bearing enables uniform and essentially jerk-free guidance of the light guide, and enables locally accurate positioning of the light guide at the points to be exposed to light on the eye.

According to advantageous embodiments of the ophthalmic device, the support body has a first conical or tapered surface which forms a slide bearing surface for a complementary second conical or tapered surface formed on the guide body, wherein the second conical or tapered surface rests or abuts on the first conical or tapered surface. Cone or cone surfaces are particularly advantageous, as they enable self-centering of the sliding surfaces relative to one another and thus of the guide body relative to the support body. In particular, it can be achieved that the slide bearing surfaces are quasi-automatically centered or optimally aligned with each other during a sliding movement. This is particularly advantageous with regard to precise guidance of the free end of the laser light guide along the path.

According to a further advantageous embodiment of the ophthalmic device, the support body and/or the guide body are/is essentially designed as a ring body or disk body. Preferably, both bodies are designed as ring bodies or disk bodies, in particular as ring bodies. Ring bodies or disk bodies may be rotationally symmetrical with respect to a ring or disk axis, whereby the size of the ring body or disk body, in particular in the case of the support body, is preferably adapted to the size of the eye to be treated. The ring bodies or disk bodies are preferably arranged essentially parallel to each other. Ring bodies or disk bodies enable, for example, a comparatively simple implementation of slide bearing surfaces, in particular axial slide bearing surfaces, for example based on cone or cone surfaces.

In embodiments, the slide bearing surface of the support body may be formed on the second side of the support body facing away from the first side. For example, in such a way that the guide body may be placed on or inserted into the support body from the second side, with the slide bearing surface preferably being formed on the side of the support body facing the guide body in the assembled state.

Corresponding or complementary slide bearing surfaces are designed in particular in such a way that they lie flat against each other in the assembled state. The structure of the slide bearing surfaces is preferably flat, so that low coefficients of friction are advantageously obtained.

According to embodiments of the ophthalmic device, the first and/or second cone surface or cone surface is/are aligned coaxially to the ring body or disk body axis of the respective ring body or disk body. Corresponding cone or cone surfaces may, for example, be designed in such a way that their common axis may be positioned coaxially to the eye axis of the eye to be treated when the support body is in place. The retaining element or fixing element on the guide body may be set up in such a way that when the guide body is moved, in particular when the guide body is rotated about the common axis, the free end of the light guide that is guided therewith is, for example, guided along a path that runs, at least in sections, concentrically to the common axis.

According to embodiments of the ophthalmic device, the first cone or cone surface is tapered towards the first side. For example, the first cone or cone surface may extend from the second side towards the first side, wherein a diameter or radius defined by the cone or cone surface becomes smaller towards the first side. In particular, the first cone or cone surface may taper in a funnel shape from the second side towards the first side. The cone surface may, for example, run around an aperture extending from the second side to the first side. The aperture may be centered or concentric to the support body, especially if the support body (without the grip element or grip part) is designed as an annular disc. Preferably, the cone axis or cone axis runs parallel to a disk or ring axis of the support body. Further preferably, the cone axis or cone axis is identical to the ring axis or disk axis. In particular, the support body with cone or cone surface may be rotationally symmetrical to the disk or cone axis.

According to embodiments, the first cone or cone surface may be aligned towards the central axis or central axis of the support body, which runs perpendicular to the second side.

In particular, the support body may be ring-shaped, with a first cone surface oriented towards the ring axis and concentric to the ring axis, which tapers from the second to the first side. The cone surface may be part of an essentially funnel-shaped, central aperture or define such an aperture. A disc-shaped support body may have a corresponding conical surface that is concentric to the disc axis or central axis of the support body. In this case, the conical surface may be part of a cone-like depression or form such a depression that tapers from the second side towards the first side.

In embodiments, the second cone or cone surface, which is designed to complement the first cone surface to form a slide bearing, may be formed on a circumferential, in particular outer, edge surface of the guide body, in particular if the latter is ring-shaped or disk-shaped.

The outer contour of the support body, even if it is ring-shaped or disc-shaped, does not have to be strictly circular, but may also have other shapes, e.g. elliptical, etc.

The guide body may be inserted from the second side into a correspondingly formed funnel-shaped opening or into a correspondingly formed cone-like recess, whereby the second cone or cone surface comes into contact with the first cone or cone surface as a sliding surface. In this case, the support body may form a first, in particular radially outer, bearing shell with respect to the slide bearing, and the guide body may form a complementary, in particular radially inner, second bearing shell with respect to the slide bearing.

Preferably, the support body and the guide body are flush with each other at least on the second side. In the area or on the first side, the guide body is preferably set back relative to the support body in such a way that, when the support body is placed on the eye, the guide body does not touch the eye, in particular when performing a movement of the guide body, such as a rotation of the guide body based on the slide bearing.

In particular, such embodiments may be assembled and mounted comparatively easily. The components, i.e. the support body and the guide body, may also be manufactured comparatively easily. Furthermore, a comparatively smooth movement of the guide body may be achieved, for example such that the guide body may be rotated relative to the support body by a user using the light guide as a manipulator.

Possible materials for the components of the ophthalmic device include plastic materials and/or metals, or composite materials made of plastic and metal.

According to embodiments, the retaining element or fixing element is set up and designed in such a way that the free end of the light guide is inclined at a predetermined angle to the central axis of the guide body or to the central axis of the cone surfaces. The angle is preferably between 35 and 45 degrees, in particular approximately or substantially 40 degrees. Such angles are particularly suitable for irradiating the ciliary muscle in connection with the treatment of glaucoma. A treatment of an eye may provide for the support body to be positioned on the eyeball, for example such that the central axis or central axis of the support body coincides with the axis of the eye. If, for example, the guide body is designed as a ring concentrically inserted into the support body, the free light guide coupled to the retaining element or fixing element may be moved along a circular path by rotating the guide body relative to the support body. Accordingly, the eye may be exposed to light, in particular light pulses, preferably laser pulses, e.g. locally along the circular path. For the treatment of glaucoma and/or for irradiating the ciliary muscle with light or laser light, the radius of the circular path is set up in such a way that the free end of the light guide may be positioned for irradiating the ciliary muscle when the support body is in place. If other parts of the eye are to be irradiated, suitable radii and/or paths, in particular circular paths, may be used.

If, in embodiments, the retaining element or fixing element is designed as a channel, a longitudinal axis of the channel may be inclined at the specified angle to the central axis.

The predetermined angle allows the eye to be exposed to light at a defined angle of incidence, with the guide body simultaneously defining the angle and the path for the free end of the light guide.

In preferred embodiments, the slide bearing surfaces and the channel are designed and arranged in such a way that the channel runs essentially parallel to the slide bearing surfaces. This means that the slide bearing surfaces may be inclined at the same angle to the central axis as the free end of the light guide held by the retaining element or fixing element.

According to embodiments of the ophthalmic device, it may be provided that the guide body is coupled to the support body in a loss-proof manner. For example, the two bodies may have complementary coupling elements (or: coupling structures), such as snap-in surfaces, complementary snap-in connections or snap elements, for example in the form of a tongue and groove connection. The coupling elements are preferably formed in one piece on the support body and guide body. Particularly preferably, the coupling elements (or: coupling structures) are formed in the area of the bearing surfaces, in particular slide bearing surfaces, e.g. in the form of coupling elements (or: coupling structures) within the respective bearing surfaces. The coupling elements or structures may be designed in such a way that when the guide body is connected or inserted into the support body, the mutual coupling is formed at least when the bearing surfaces are in contact with each other as intended. A coupling is particularly advantageous in that when the guide body is moved, for example by the light guide as a manipulator, the mutual position of the bearing surfaces or slide bearing surfaces remains the same, i.e. it may be ensured that the angle defined by the retaining element or fixing element remains essentially the same and is not changed by accidental slipping or tilting of the guide body in relation to the support body.

According to embodiments of the ophthalmic device, the guide body may, as already indicated, be movably attached to or coupled to the support body in such a way that a movement of the guide body and an accompanying movement of the retaining element or fixing element relative to the support body may be effected by a light guide held on or by the retaining element or fixing element, e.g. a light guide inserted into the channel. According to embodiments, the guide body may be mounted on the support body in such a way that the end of the light guide held on or by the retaining element or fixing element and facing the first side follows a circular path when the guide body moves relative to the support body. The circular path may, for example, have a diameter in the range between 15 mm and 25 mm, preferably between 17 and 19 mm, in particular around 18.75 mm.

According to embodiments of the ophthalmic device, it further comprises a device for engaging and retaining the eyelids of the eye when the support body is placed on the eyeball. The device may, for example, be designed in the manner of an eyelid speculum, e.g. on opposite lateral sides and/or laterally circumferentially on the support body. In particular, the device may be formed on the support body, for example in one-piece or multi-piece form.

In embodiments, the ophthalmic device may comprise exactly two or more than two retaining elements or fixing elements, in particular channels. The retaining elements or fixing elements may, for example in the case of ring bodies or disk bodies, be arranged in pairs, e.g. opposite each other with respect to the central axis or central axis, preferably equally distributed in the circumferential direction. In particular, in the case of two or more retaining elements or fixing elements or pairs, these may each be set up or used for an angular range of rotation for the movement of the guide body. In the case of two retaining elements or fixing elements, the angular range of rotation may be approximately 180°, in the case of four retaining elements or fixing elements, the respective angular range of rotation may be, for example, 90° and so on. In embodiments, it is possible that the retaining elements or fixing elements are arranged on the guide body in such a way that they may be transferred into one another by rotating the guide body about the central axis or central axis. This allows, for example, a full-angle rotation (360°) to be implemented.

In embodiments, one of the coupling elements may be formed as an annular or latching bead in the slide bearing surface of the contact body or the guide body and a complementary annular groove or latching groove may be formed in the slide bearing surface of the guide body or the contact body. Preferably, the complementary coupling elements are designed in such a way that, at the latest when the slide bearing surface of the guide body is brought into contact with the slide bearing surface of the contact body, the complementary coupling elements lock together. The latching groove or the latching bead extend, preferably locally, parallel, e.g. circularly, to the slide bearing surfaces, so that the guide body may be moved in accordance with the slide bearing surfaces and the guide body is nevertheless held loss-proof on the contact body.

In embodiments, the diameter of the channel may be in the range between 0.3 mm and 6 mm, preferably between 0.4 mm and 0.55 mm, in particular around 0.48 mm.

In embodiments, the guide body may be designed as an annular body with a minimum outer diameter in the range from 15 mm to 20 mm, in particular about 18.75 mm, and with a maximum outer diameter in the range from 25 mm to 30 mm, in particular about 27.59 mm. In particular, the guide body as a whole may be designed in the manner of a funnel or funnel-shaped cone, whereby radially inner and radially outer cone surfaces may run essentially parallel to each other.

In embodiments, the support body with grip element may be shaped like a spoon, for example in that the grip element extends obliquely upwards with respect to the second side. Such a grip element is advantageous, for example, with regard to the manual positioning of the support body on the eye and with regard to the movement, in particular rotation, of the guide body by the light guide as a manipulator.

Similar to the guide body, the support body may be funnel-shaped with conical inner and outer surfaces, preferably running essentially parallel to each other.

Dimensions for the length of the grip element may be in the range between 50 mm and 80 mm. Dimensions for the minimum outer diameter of the support body may be between 15 mm and 20 mm. Dimensions for the maximum outer diameter can be between 25 mm and 33 mm. The height of the support body measured parallel to the central axis or central axis may be in the range of 5 mm to 8 mm, in particular about 6.8 mm to 7 mm.

The invention is explained further below with reference to exemplary embodiments. Reference is also made to the drawings.

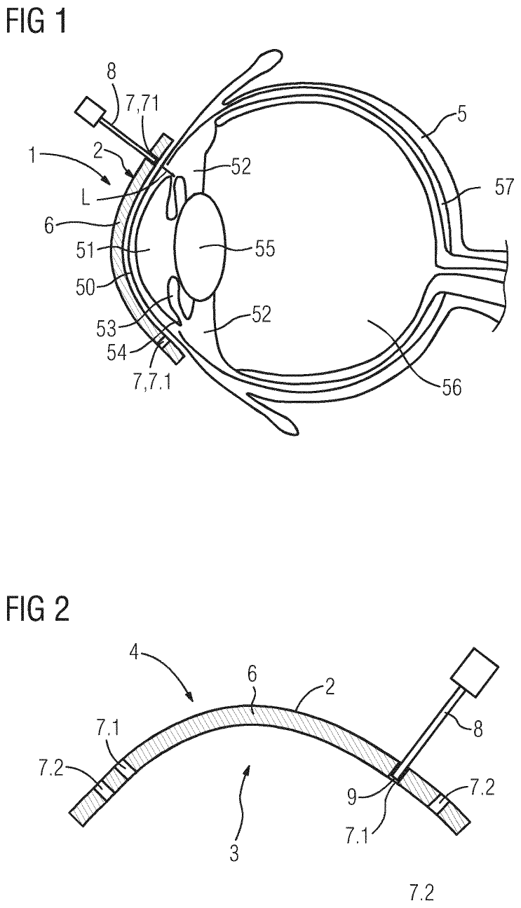

FIG. 1 shows an ophthalmic mask placed on an eyeball of an eye with a light guide positioned in a positioning marker;

FIG. 2 shows a mask body of an ophthalmic mask according to FIG. 1 in section;

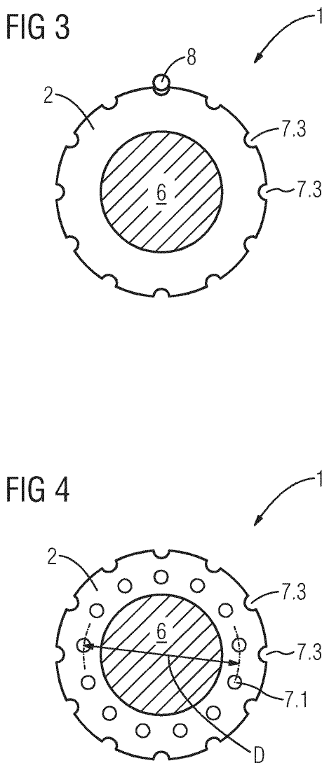

FIG. 3 shows a first example of an ophthalmic mask;

FIG. 4 shows a second embodiment of an ophthalmic mask;

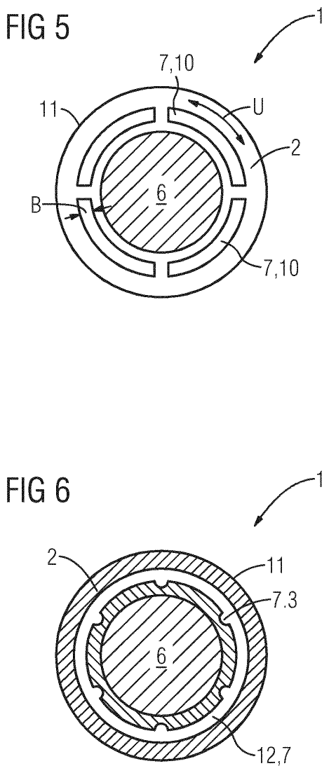

FIG. 5 shows a third embodiment of an ophthalmic mask;

FIG. 6 shows a fourth embodiment of an ophthalmic mask;

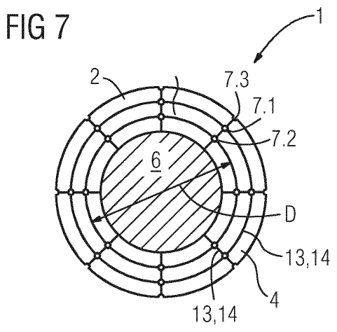

FIG. 7 shows a fifth embodiment of an ophthalmic mask;

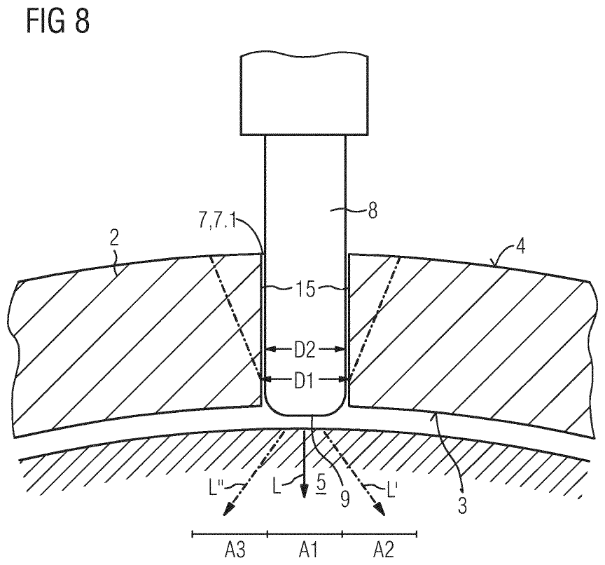

FIG. 8 shows a detailed illustration of the use of the ophthalmic mask in laser treatment of the eye;

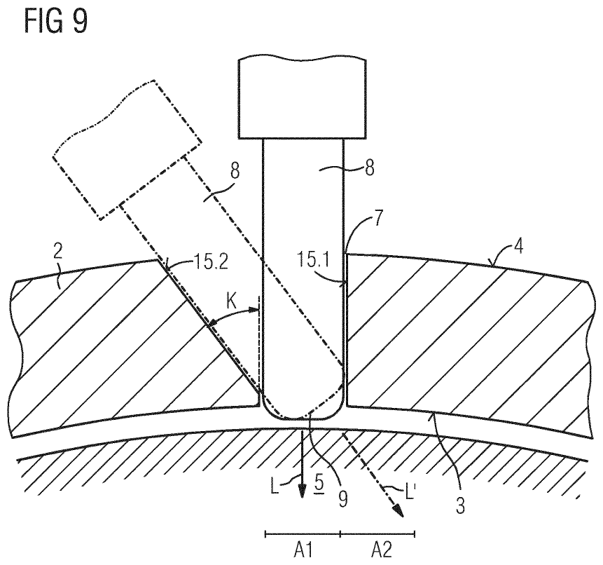

FIG. 9 shows an additional detailed illustration of the use of the ophthalmic mask during laser treatment of the eye;

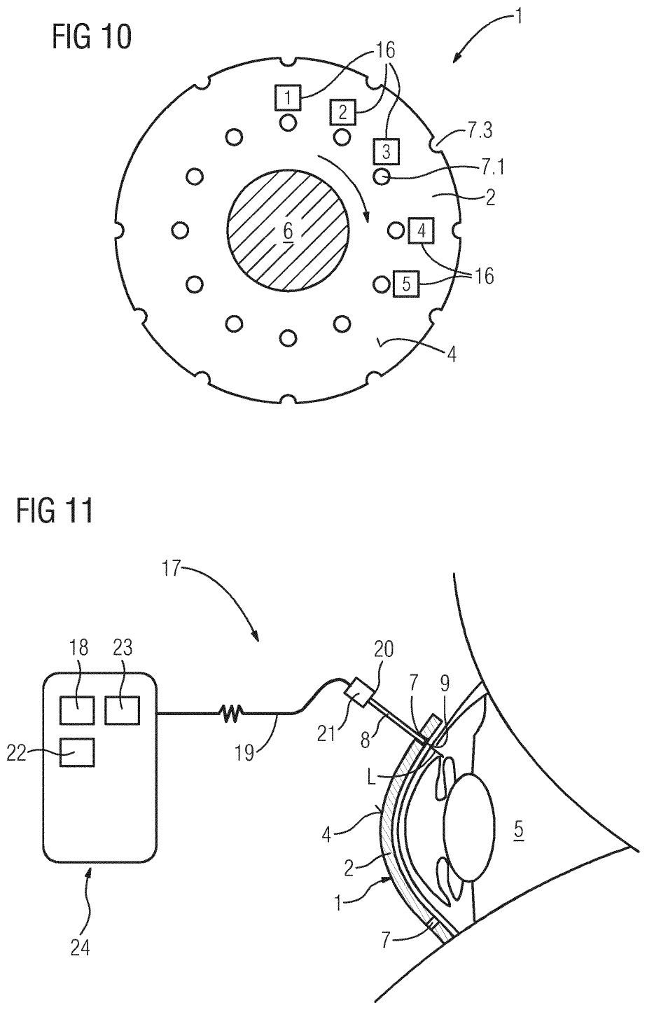

FIG. 10 shows a sixth embodiment of an ophthalmic mask; and

FIG. 11 shows an embodiment of an ophthalmic device comprising an ophthalmic mask;

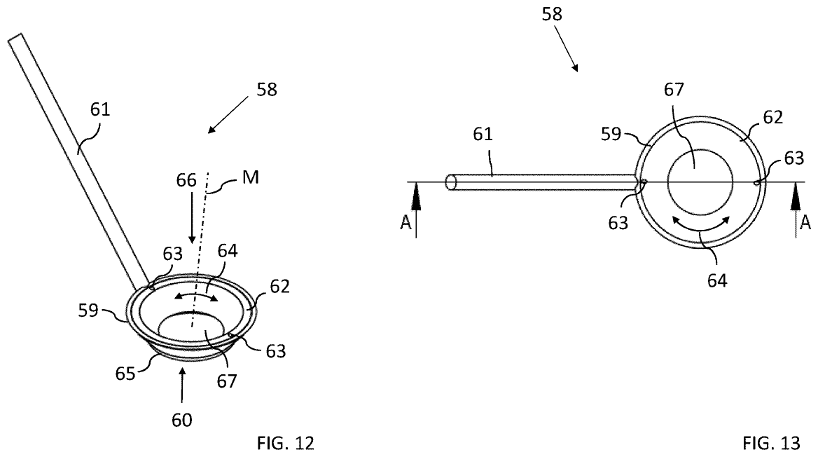

FIG. 12 shows an embodiment of another ophthalmic device;

FIG. 13 shows a top view of the device of FIG. 12;

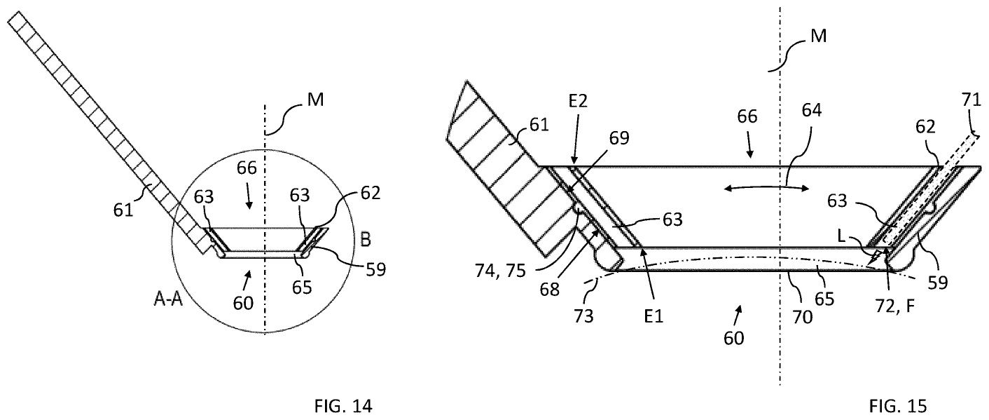

FIG. 14 shows a sectional view according to A-A of FIG. 13;

FIG. 15 shows a detail according to B of FIG. 14;

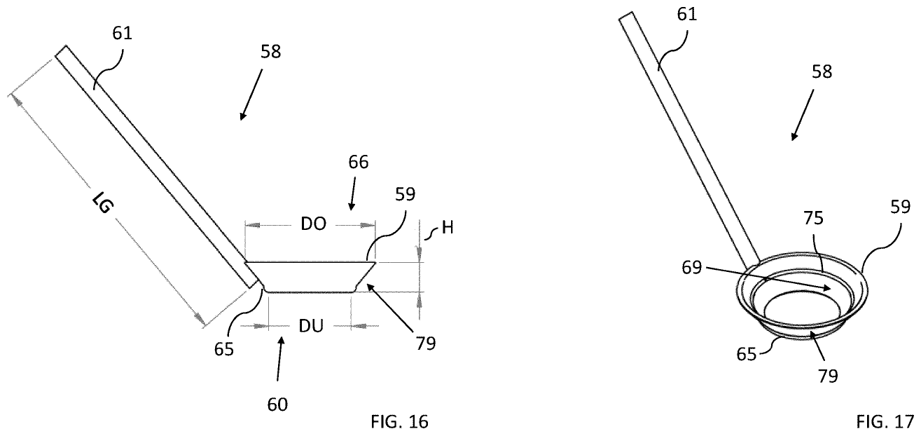

FIG. 16 shows the device of FIG. 12 with dimensions;

FIG. 17 shows a perspective view of a support body with grip element according to the device of FIG. 12;

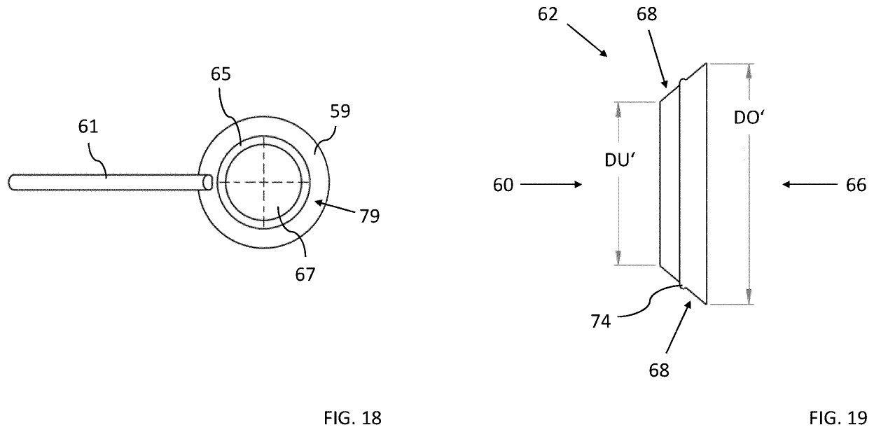

FIG. 18 shows a view of FIG. 17 from below;

FIG. 19 shows a side view of a guide body of the device according to FIG. 12;

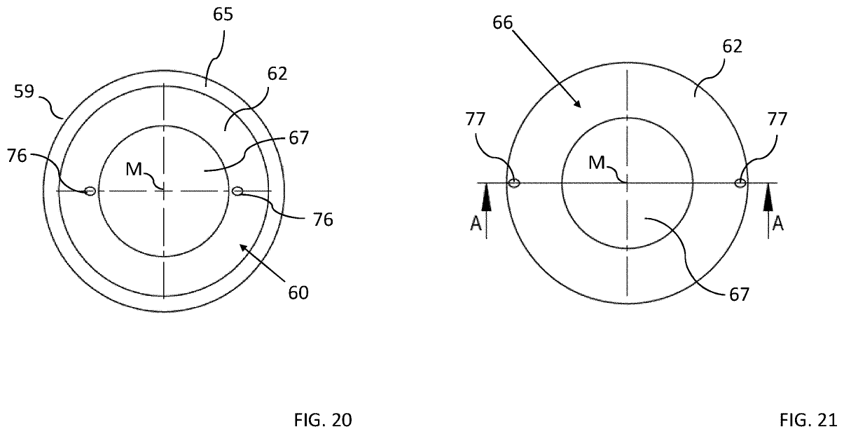

FIG. 20 shows a view of the support body and guide body of the device according to FIG. 12 from below;

FIG. 21 shows a top view of the guide body;

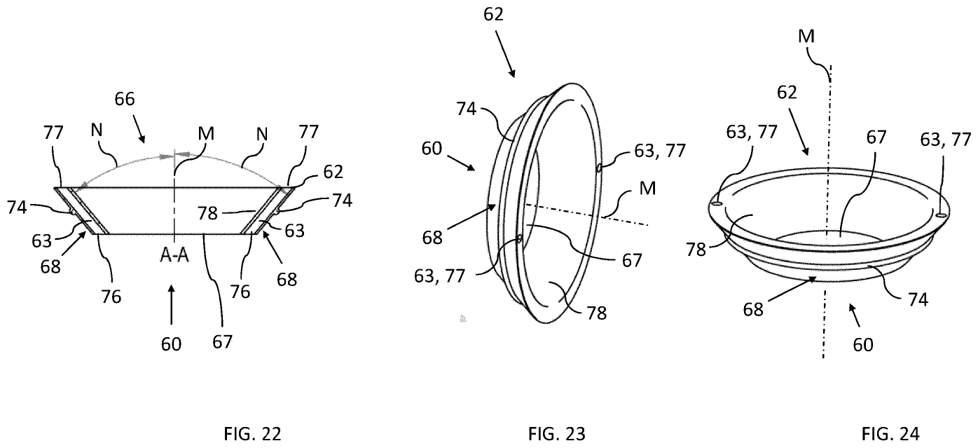

FIG. 22 shows a sectional view according to A-A of FIG. 21;

FIG. 23 shows a perspective view of the guide body;

FIG. 24 shows an additional perspective view of the guide body; and

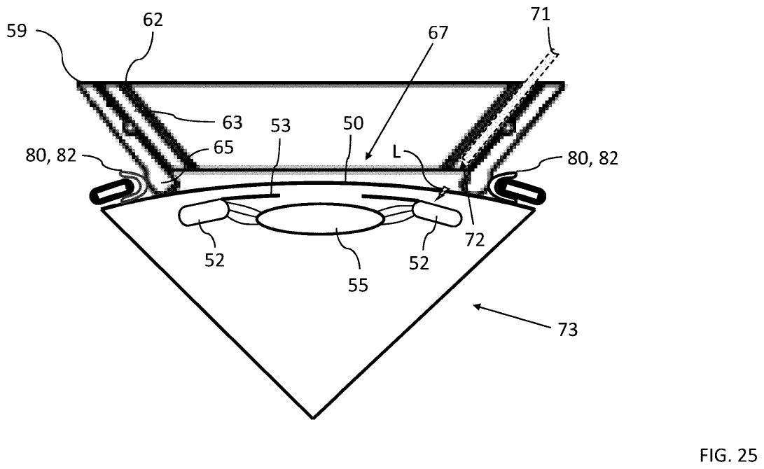

FIG. 25 schematically shows a use case of the device of FIG. 12.

Unless otherwise indicated, corresponding parts and elements are marked with the same reference signs in the figures. Scales between the figures may vary.

FIG. 1 shows an eye 5 with cornea 50, the anterior eye chamber 51 filled with ocular fluid, the ciliary muscle 52, the iris 53, the trabecular meshwork 54, the lens 55 held by the ciliary muscle 52, the vitreous body 56 and the retina 57 located in the posterior eye and other undesignated parts.

An ophthalmic mask 1 is positioned or applied to the eye 5 in the area of the cornea 50. As can be seen in the sectional view of FIG. 1, the ophthalmic mask 1, hereinafter also referred to as mask 1 for short, has a shell-like design and is adapted in shape to the shape of the eye 5 at least in the region of the cornea 50. Consequently, the mask 1 comprises a shell-like mask body 2 for positioning on or on the eye 5, in particular in the area of the cornca 50.

FIG. 2 shows a section through a mask body 2 of an ophthalmic mask 1. This mask body 2 as well as the mask bodies described further below may be positioned on or on an eye 5 in the area of the cornea 50 as shown in FIG. 1.

The mask body 2 of FIG. 2 is shown in a curved shape adapted to the shape of the eye 5. The mask body 2 may be flexible or rigid, in particular with regard to its curvature.

The mask body 2 has a first side 3 for contacting or resting on the eye 5, i.e. the eyeball of the eye 5. In addition, the mask body 2 has a second side 4, which faces away from the eye 5 when positioned on the eye 5.

The mask body 2 has a flat, in top view (analogous, for example, to FIG. 3) for example circular, central area 6. In use, the mask body 2 or the mask 1 is positioned at or on the eye 5 in such a way that the central area 6 covers at least the iris 53 and the pupil of the eye 5 enclosed by it. The central area 6 may be adapted to a respective outer diameter of the iris 53 of the eye 5. For example, mask bodies 2 with different diameters for the central area 6 may be provided for different iris sizes, so that a suitable mask 1 may be used for the laser treatment of the eye 5.

The mask 1 comprises a plurality, in particular a plurality, of positioning markers 7. The positioning markers 7 are designed and arranged to be visible at least from the second side 4 of the mask 1, for example to a person performing the laser treatment, for example a selective laser trabeculoplasty.

The positioning markers 7 are designed in such a way that a light guide 8 may be positioned therein or thereon during laser treatment. In the example of FIG. 1, it is shown that the light guide 8 is positioned in a positioning marker 7, which in the example of FIG. 1 is formed as a recess, in particular as a through hole. During laser treatment of the eye 5, the eye 5 is irradiated with laser light L, for example with laser pulses, via the light guide 8.

The exemplary embodiments describe the special case of laser treatment. In general, the masks 1 may also be used with a different type of light as described below.

In the example of FIG. 1, the positioning marker 7 with the light guide 8 placed therein is positioned such that laser light L is emitted or irradiated into the area of trabecular meshwork 54. The effect of the laser light L, in particular in the form of laser pulses, improves the outflow of aqueous humor via the trabecular meshwork 54 and the outflow channel in the chamber angle of the eye. The improved aqueous humor outflow can reduce the intraocular pressure that causes glaucoma. Thus, if the positioning markers 7 of the mask 1 are arranged in such a way that they are located in the area of the trabecular meshwork 54 when the mask 1 is placed on the eye 5, the eye 5 may be selectively irradiated locally by selective laser application at the positioning markers 7, for example in the context of glaucoma treatment. The decisive feature of the mask 1 proposed herein is in particular that the light guide 8 is or may be positioned in the positioning mark 7, and the laser light L may be emitted directly onto the respective treatment area defined by the positioning mark 7. Direct and immediate irradiation is particularly advantageous compared to the method of WO 2018/152020 A1, since interferences due to interfaces and/or refraction may be avoided.

The shell-like mask body 2 is not permeable to the laser light L in the central area 6. Furthermore, the mask body 2 is also not permeable to the laser light L (opaque) outside the central area 6, whereby the mask body is only permeable to the laser light L at the positioning markers 7, so that laser light L may be applied to the eye 5 via the light guide 8 positioned in or at the positioning markers 7.

By positioning the light guide 8 in a positioning marker 7 and applying laser radiation, a locally selective laser treatment of the eye 5 can thus be performed.

As can be seen from FIG. 1, the area size of the central area 6 is adapted to the area size or diameter of the iris 53 of the eye 5 to be treated in the respective application. In other words, the mask 1 is selected so that the central area 6 covers the iris 53. Consequently, when the mask 1 is in place, the laser light may be prevented from entering the eye 5 via the iris 53, pupil and lens 55. This is particularly necessary to prevent laser light L from hitting the retina 57 and causing damage.

The positioning markers 7 are arranged radially outside the central area 6. The positioning markers 7 are arranged such that, as already described, when the light guide 8 is positioned in a positioning marker 7, the eye 5 for each positioning marker 7 may be selectively exposed to laser light L emitted by the light guide 8 as such.

In the example of FIG. 1, the positioning markers 7 are designed as recesses 7.1, for example in the form of through-holes. The positioning markers 7 of FIG. 1 are arranged on a circle around the central area 6, as shown, for example, in the embodiment according to FIG. 4.

In the exemplary embodiment of FIG. 2, first and second positioning markers 7 are formed as recesses 7.1 and 7.2, which are each arranged on a circle around the central area 6. The light guide may be placed in these positioning markers 7, as shown in FIG. 2 as an example for one of the positioning markers 7.

FIG. 3 shows a further exemplary embodiment in which positioning markers 7 are formed as face-sided indentations 7.3. The indentations 7.3 are adapted in their shape and cross-sectional geometry to that of the light guide 8 to be used. In particular, the light guide 8 may be inserted into the indentations 7.3 and placed against the inner edges of the face-sided indentations 7.3. Compared to recesses 7.1 and 7.2, indentations 7.3 may have the advantage that the light guide 8 may be positioned more easily in an indentation 7.3 and guided to the next indentation 7.3 after laser treatment, for example by inserting it at the edge and sliding it along the face-sided edge of the mask 1.

In addition to the indentations 7.3, the mask 1 shown in FIG. 3 may also have recesses 7.1 or 7.2, corresponding to the recesses of FIG. 1 or 2, which may be arranged in a circle around the central area 6.

The indentations 7.3 and recesses 7.1 and 7.2 have the common feature that they are adapted in shape and geometry, e.g. diameter or opening radius, to the geometry, e.g. the diameter, of the light guide 8. More precisely, the geometry of the positioning markers 7 is adapted to the geometry of the distal end 9 of the light guide 8, which is set up to be positioned in the positioning markers 7, i.e. in the indentations 7.3 and recesses 7.1, 7.2, during laser treatment of the eye 5.

If individual areas of the eye 5 are to be treated with different light guides 8, the geometry of positioning markers 7 may be specifically adapted to the light guide 8 to be used.

FIG. 4 shows an exemplary embodiment in which two groups of positioning markers 7 are present. One group is formed by indentations 7.3 on the face side, which are distributed around the circumference of the mask body 2. A second group is formed by recesses 7.1 which are arranged in a circle around the central area 6. Further groups of recesses may be present, for example on a circle with a larger or smaller radius than that of the second group, but all outside the central area 6.

Preferably, the indentations 7.3 and the recesses 7.1 are arranged essentially equally distributed to one another in the circumferential direction.

The positioning markers 7, whether in the form of recesses 7.1, 7.2 or indentations 7.3, may be used to predetermine treatment points for a person performing the laser treatment, whereby, in particular, a laser treatment on the eye 5 may be performed reliably. For example, the positioning markers 7 may be arranged in such a way that an optimal exposure of the trabecular meshwork 54 may be achieved during selective laser trabeculoplasty with the distal end 9 positioned in the respective positioning markers 7. Furthermore, laser treatment may be simplified because the positioning markers 7 also provide an orientation aid for the person performing the treatment as to where the light guide 8 should be selectively placed on the eye 5. The positioning markers 7 may be arranged in a specific pattern for a particular laser treatment.

The distances between adjacent positioning markers 7, whether positioning markers 7 within a group or different groups, are preferably selected so that the area to be treated, e.g. the trabecular meshwork 54, may be exposed to a sufficient amount of laser light L, in particular defined laser pulses, without affecting areas excessively by repeated irradiation. Consequently, in addition to reliable treatment, gentle laser treatment may also be achieved.

In the arrangement of the positioning markers 7 shown in FIG. 4, the indentations 7.3 and the recesses 7.1 are offset with respect to each other with respect to the circumferential direction. The arrangement and the spacing of the positioning markers 7 may, for example, be selected such that during selective irradiation by each of the positioning markers 7, the tissue area of the eye 5 to be treated, e.g. the trabecular meshwork 54, is exposed to laser light L in a respectively predetermined area fraction.

Apart from the circular arrangement of a group of positioning markers 7 shown in FIG. 4, a group of positioning markers 7 may also be arranged according to a different geometric pattern, for example along an ellipse extending around the central area.

In the embodiments of FIGS. 1 to 4, the indentations 7.3 may have a cross-section in the form of a circular segment, for example, and the recesses 7.1, 7.2 may have a cross-section in the form of a circular area. A radius assigned to the circular segment or circular area is adapted to the radius of the distal end 9 of the light guide 8 provided for the punctual application of laser light, so that the distal end 9 may be inserted into a respective recess 7.1, 7.2 or indentation 7.3. Or, in other words, the radii are adapted so that the recesses 7.1, 7.2 or indentations 7.3 may each receive the distal end 9.

To simplify the positioning of the distal end 9 in a positioning marker 7, the latter may have slightly larger radii than the distal end 9, so that insertion and removal of the distal end 9 does not cause any significant force effects on the mask 1. With regard to the geometry of the positioning markers 7, it is possible that these, specifically the recesses 7.1, 7.2 on the second side or the indentations 7.3 on the face side, are specially shaped, for example conically shaped, in order to facilitate insertion and positioning of the distal end 9.

The exemplary embodiment of FIG. 5 shows an ophthalmic mask 1 in which positioning markers 7 are provided in the form of notches 10. The notches 10 extend in the circumferential direction U around the central area, and are designed as elongated slots in the circumferential direction U. A width B of the notches or slots 10 is adapted to the diameter of the distal end 9 of the light guide 8. During laser treatment, the distal end 9 may thus be inserted into a respective notch 10 and displaced in the circumferential direction U over the length of the notch 10. With simultaneous application of laser light L or laser pulses, the area of the eye 5 located under the notch 10 may be treated. Compared to individual recesses 7.1, 7.2 or indentations 7.3, slits may allow the laser treatment to be carried out quickly, as the distal end 9 does not have to be specifically positioned as often. However, it may also be achieved here that the laser light L can essentially be delivered directly from the distal end to the eye 5.

Corresponding to the notches 10 shown in FIG. 5, which are located inside the outer face-sided edge 11 of the mask body 2, notch-shaped indentations may be present running along the face-sided edge 11. The distal end 9 of the light guide 8 may be placed in these and moved along the face-sided edge 11 in the circumferential direction during laser treatment.

The notch- or slot-shaped positioning markers 7 of FIG. 5 are not limited to the specifically shown number, arrangement and their orientation in the circumferential direction. Furthermore, there may also be circular recesses 7.1, 7.2 and/or face-sided indentations 7.3, e.g. in the form of segments of a circle, as shown in FIGS. 1 to 4.

FIG. 6 shows an embodiment in which a positioning marker 7 is formed as a circular ring 12. In contrast to FIG. 5, the circular ring 12 runs completely around the central area 6 and is not, as in FIG. 5, interrupted by radial webs. In the circular ring 12 shown in FIG. 6, the radially outer edge is smooth and the radially inner edge has indentations 7.3, the shape of which may, for example, correspond to FIG. 3 or 4. Alternatively, the radially inner edge of the circular ring 12 may also be smooth and/or the radially outer edge may have indentations. When performing a laser treatment, the distal end 9 of the light guide 8 of the mask 1 of FIG. 6 may be placed in the circular ring 12 and, for example, guided in a continuous movement in the circumferential direction in the circular ring 12 when bearing against the radially outer edge. It is also possible for the distal end 9 of the light guide 8 to be placed in the circular ring and, for example, to be guided successively from indentation 7.3 to indentation 7.3 in a discontinuous movement when bearing against the radially inner edge in the circumferential direction of the circular ring 12. Between the indentations 7.3, the inner and/or outer edge may be used as a guide. In embodiments, the mask 1 of FIG. 6 may also have positioning markers 7 of a different geometry, e.g. recesses 7.1, 7.2 and/or end-face indentations 7.3, in addition to the circular ring 12.

To implement a positioning marker 7 in the form of a continuous circular ring 12, the mask body 2 may, for example, be constructed with at least two layers. A first layer forming the first side 3 may be designed as a continuous flat layer, e.g. made of glass. The layer components forming the second side 4 could then be glued to the first layer, whereby the layer components are shaped in such a way that, when placed accordingly, they cover the first transparent layer while leaving the circular ring open.

FIG. 7 shows an embodiment in which the recesses 7.1, 7.2 and the indentations 7.3 are arranged radially outside the central area 6 in the manner of a cobweb pattern. Corresponding patterns may contribute in particular to achieving an advantageous mechanical stability of the mask body 2. Unlike the illustration in FIG. 7, in the cobweb pattern, the groups of recesses 7.1, 7.2 and the indentations 7.3 may, in the circumferential direction, be arranged offset from one another as well, so that the recesses 7.1, 7.2 and the indentations 7.3 are not arranged along a radial line extending from the center of the central area 6. For example, the positioning markers 7 (7.1, 7.2, 7.3) may be arranged in a direction outwards from the central area along curved curves or circle tangents, wherein circule centers assigned to the circle tangents may coincide with the center of the central area 6, for example.

In the exemplary embodiment of FIG. 7, and also the other exemplary embodiments, groups of positioning markers 7 are each arranged on circular lines which are concentric with the central area. A circle diameter D (FIG. 7, FIG. 4) of such a circle may, for example, be in the range from 9.5 mm to 13 mm, in particular in the range from 10 mm to 12 mm.

As indicated in FIG. 7, the mask body 2 may have a groove structure 13 on the second side 4. The groove structure may have a plurality of grooves 14, for example in the form of depressions, each extending between two positioning markers 7. The grooves 14 may be implemented on the second side 4 by removing material or in some other way, with the groove depth being selected for a given thickness of the mask body 2 such that the latter is opaque to the laser light L in the area of the grooves 14. A groove structure 13 may generally be present between recesses 7.1, 7.2 and/or indentations 7.3, wherein a groove 14 connecting two recesses 7.1, 7.2 or a recess 7.1, 7.2 and an indentation 7.3 may be set up as a guide aid for the distal end 9 of the light guide 8.

FIG. 8 shows an enlarged view of the mask body 2 in the area of a positioning marker 7 formed as recess 7.1. The positioning marker 7 of the exemplary embodiment is formed as a through hole with an essentially cylindrical inner wall 15. In the illustration of FIG. 8, the distal end 9 of a light guide 8 is inserted into the through-hole and positioned for direct delivery of laser light L, in particular laser pulses, into the eye 5 for its treatment.

In order to make it easier for a user to insert the distal end 9 into the through hole, the inner wall 15 may have a conical chamfer, at least in sections. The diameter D1 of the through hole may decrease from the second side 4 towards the first side 3, with a minimum diameter D1 in the region of the first side 3 being adapted to the diameter D2 of the distal end 9. The minimum diameter D1 essentially corresponds to the diameter D2 of the distal end 9.

Conical beveled inner walls 15 are indicated by dashed lines in FIG. 8. The conical chamfer may be provided for the entire inner wall 15. It is also possible that the conical chamfer is only formed on one side or a limited area of the inner wall 15 of the through hole.

In embodiments, the conical bevel may be designed in such a way that the light guide 8 positioned in the through hole may be tilted within certain limits. The possibility of tilting the light guide 8 makes it possible to extend the treatment area A1 that may be exposed to laser light L, for example, only perpendicular to the mask body 2. Two further treatment areas A2 and A3 are shown schematically in FIG. 8, which may be reached by tilting the light guide 8 and irradiating laser light L′ and L″ at a corresponding angle.

FIG. 9 shows an embodiment in which a positioning marker 7 in the form of a through-hole is beveled in a conical shape on only one side of the inner wall 15. The chamfer formed on one side also serves as an insertion aid for the distal end 9. A first treatment area A1 (laser light L) may be reached when the light guide 8 is positioned or placed against the inner wall 15.1, which runs essentially perpendicular to the second side 4. A second treatment area A2 (laser light L′) may be reached by positioning the light guide 8 against the inner wall 15.2, which extends at an angle to the second side 4. It is therefore possible to enlarge the accessible treatment area by means of corresponding conical bevels, whereby a selective selection of local treatment areas is still possible by means of the positioning marker 7.

Bevels may be present not only for through holes, but are also suitable for all the shapes and geometries of the positioning marks 7 mentioned herein, in particular for slot-shaped and circular recesses as well as for indentations, in order to extend the possible treatment area.

FIG. 10 shows an embodiment in which visual markings 16 assigned to the positioning marks 7.1 to 7.3 are provided or attached on the second side 4 of the mask body 2, i.e. facing the person performing the treatment in the application case. The sequence of punctual irradiation may be predetermined by such markings 16. In FIG. 10, corresponding markings 16 are indicated only for some of the position marks 7.1. With the markings 16 shown in FIG. 10, a clockwise treatment sequence is indicated by way of example.

FIG. 11 shows an ophthalmic device 17, comprising an ophthalmic mask 1, for example formed according to one of the embodiments described herein in accordance with the invention. Furthermore, the device 17 comprises a laser light source 18, and a light guide 8 connected to the laser light source 18 via a connection line 19 and having a free distal end 9, wherein a handle 21 is provided at a proximal end 20 of the light guide 8. The combination of handle 21 and light guide 8 may be regarded as a laser applicator. The shape, in particular the diameter, of the distal end 9 is adapted to the diameter of the positioning markers 7, so that the distal end 9 may be positioned selectively, and for example sequentially, in each of the positioning markers 7. After proper positioning, the laser light source 18 may be activated to emit laser light L, in particular laser pulses, and the eye may be selectively exposed locally and substantially directly to the laser light L emitted by the distal end 9.

The device 17 may further comprise a control unit 22 and a user interface 23. The control unit 22 may comprise one or more processors or computing units arranged, for example, to generate control signals that cause laser light L to be emitted via the laser applicator. The user interface 23 may comprise one or more control elements or control panels for setting operating parameters, e.g. the laser energy, the laser pulse length, etc.

The laser light source 18, the control unit 22 and the user interface 23 may be accommodated or integrated in a mobile or stationary device unit 24. The light guide 8 and/or the handle 21 may be connected to the device unit 24 via the connection line 19 in a fixed or attachable and detachable manner. The light guide 8 may be connected to the handle 21 in a fixed or attachable or detachable manner. The laser light source 18 may be a Nd:YAG laser source, in particular a pulsed laser source.

For a laser treatment of the eye, in particular glaucoma, the device 17 may be operated according to the following process steps:

Placing a mask 1 on the eyeball of an eye 5 in such a way that the central area 6 covers at least the iris 53 of the eye 5 and the positioning markers 7 are arranged outside the area of the iris 53.

Positioning the distal end 9 of the light guide 8 directly at or in one of the positioning markers 7 of the mask 1.

Application of laser light L, in particular laser pulses, into a local treatment area A1, A2, A3 of the eye 5 defined by the positioning marker 7, wherein the laser light L is emitted according to a predetermined energy, pulse length and/or frequency.

Preferably, the method comprises the further steps:

Removing the distal end 9 of the light guide 8 from the positioning marker 7 and repositioning the distal end 9 in another, different positioning marker 7 of the mask 1.

Application of laser light L, in particular laser pulses, into a further local treatment area of the eye 5 defined by the other positioning marker 7, the laser light L being emitted in accordance with a predetermined energy, pulse length and/or frequency.

Steps d) and c) are preferably repeated until an area of the eye 5 to be treated is locally selectively exposed to laser light L via the positioning markers 7. For example, steps d) and c) may be repeated until the eye 5 is locally selectively exposed to laser light L at least once via a predetermined group of positioning markers 7, in particular via all positioning markers 7.

The ophthalmic mask proposed herein is particularly adapted for use in combination with a laser applicator having a light guide with a free distal end, and in which the shape, in particular the diameter, of the free distal end is adapted to the shape, in particular the diameter, of the positioning markers in such a way that the free distal end may be selectively positioned directly at or in a positioning marker of a group of positioning markers, preferably directly at or in all positioning markers, so that a treatment area of the eye defined by a respective positioning marker may be selectively exposed locally essentially directly to the laser light emitted at the distal end.

In this respect, the underlying invention also comprises, in particular, a combination of an ophthalmic mask described herein and a laser applicator, wherein the laser applicator comprises a light guide with a free distal end, and wherein the shape and geometry of the positioning markers and the shape and geometry of the free distal end are adapted to each other in such a way that the distal end may be inserted into at least one group of positioning markers, so that after placing the mask on the eye and positioning the distal end in a positioning marker, the eye may be locally selectively exposed to laser light. The positioning markers define local treatment areas on the eye, for example the area of the trabecular meshwork in an application for the treatment of glaucoma. The underlying invention thus enables efficient and locally precise treatment of the eye with light, in particular laser light.

FIG. 12 shows an exemplary embodiment of a further ophthalmic device 58, for example according to any one of claims 11 to 22, wherein FIGS. 13 to 25 show further views and/or components and/or applications of the device.

The further ophthalmic device 58 according to FIG. 12, also referred to as device 58 for short, comprises a support body 59 with a first side 60 for direct support on an eyeball, in particular for direct support on the cornea 50 of an eye 73 (FIG. 25). The support body 59 has a grip element 61, or is connected or integrally formed with the grip element 61. The grip element 61 is for manually handling the device 58 by a user, for example an attending physician, and in particular for holding the support body 59 on the eyeball and/or pressing it against the eyeball.

The device 58 further comprises a guide body 62, which has at least one retaining element or fixing element 63 for releasably holding a free end of a light guide on the guide body 62. In the example shown, two retaining or fixing elements 63 are present, which are formed as channels 63 extending in the guide body 62 or passing through the guide body. In this respect, the terms retaining element or fixing element and channel are used synonymously below.

The channel 63 is formed and provided in the guide body 62 in such a way that, when the support body 59 is placed or positioned on the eyeball, light may be selectively applied to the eyeball or components thereof through the light guide 71 (FIG. 25).

The guide body 62 is movably mounted, in this example slidably mounted, on the support body 59 in such a way that the channels 63 move along a predetermined path when the guide body 62 moves relative to the support body 59. In the example shown, the movement is a rotation about a central axis or central axis M, in particular a circular movement. Possible rotational movements 64 are indicated by a double arrow in FIG. 12.

The support body has a ring bead 65 on the first side 60 for contact with the eyeball. The ring bead 65 is toroidal or bead-like in shape, with curves oriented towards the first side or a rounded contact surface or line for gentle contact with the eyeball or cornea 50 (FIG. 25).

FIG. 13 shows a top view of the device 58 of FIG. 12. According to a combined view of FIG. 12 and FIG. 13, and of the further FIGS. 14 ff., the support body 59 and the guide body 62 are each designed as an annular body, wherein the guide body 62 is inserted into the support body 59 from the second side 66 facing away from the first side 60, in particular according to the view of FIGS. 12 and 13 described above. Apart from a ring structure, a disk structure is also possible. A ring structure or a design of the body as a ring body has the advantage that an aperture 67, in particular a viewing aperture, is present in the center, which facilitates positioning on the eye, for example concentrically to the iris or pupil.

FIG. 14 shows a sectional view according to A-A of FIG. 13, and FIG. 15 shows a detail according to B of FIG. 14. It can be seen from the illustrations of FIGS. 14 and 15 that the support body 59 and the guide body 62 each have a conical structure tapering in a funnel shape towards the first side 60. In the assembled state, i.e. when, according to the view according to FIGS. 14 and 15, the guide body 62 is inserted into the support body 59 from above, a first conical surface 68 of the guide body 62 facing away from the central axis M and extending around the central axis M is in contact with a second conical surface of the support body 59 facing the central axis M and extending around the central axis M. The two conical surfaces 68, 69 form slide bearing surfaces between the guide body 62 and the support body 59, and enable the rotary movement 64 of the guide body 62 about the central axis M. In particular, the slide bearing surfaces form an axial bearing with conical sliding surfaces.

As can be seen in particular from FIG. 14 and FIG. 15, the channels 64 extend in the wall of the guide body 62, substantially parallel to the slide bearing surfaces. As can be seen in particular from FIG. 15, a first end E1 of the guide body 62 facing the first side 60 extends approximately as far as the ring bead 65 formed on the support body 62 on the first side 60, at least not quite as far as the support edge 70 formed on the ring bead 65 on the first side 60 for resting on the eye. This may prevent the guide body 62 from touching the eye or resting on the eye or the cornea, so that no frictional forces or grinding movements are transmitted to the eye or the cornea when the guide body 62 is rotated.

The length of the guide body 62, measured parallel to the central axis M, is selected accordingly so that the first end E1 is a defined distance away from the support edge 70, and in any case remains behind the support edge 70 when viewed from the first side 60 to the second side 66.

A second end E2 of the guide body 62 facing the second side 66 is approximately flush with the support body 59. This results in a flush or flat surface on the second side 66.

FIG. 15 further shows a light guide 71 in sections, which may for example comprise one or more cladded light guides, for example laser guides. In the example of FIG. 15, the light guide 71 is inserted into the channel 63 from the second side 66, wherein the diameter of the channel 63 substantially corresponds to the diameter of the light guide 71. The light guide 71 is inserted approximately up to the opening of the channel 63 located at the first end E1, wherein a light exit surface 72 is oriented towards the first side 60, so that light L, in particular light pulses, such as laser pulses or laser light, may be applied to the eye. In FIG. 15, the eye, specifically in the area of the pupil, is schematically indicated by a dash-dotted line 73, whereby it can be seen from this schematic illustration that the ring bead 65 rests with the support edge 70 on the eye 73 or the cornea. It can also be seen, at least schematically, that the first end E1 of the guide body 62 does not touch the eye 73 and is spaced apart from it respectively.

The slide bearing surfaces, i.e. the first and second conical surfaces 68, 69, enable revolving or rotating of the guide body 62 with very low frictional forces, whereby the friction may optionally be additionally reduced, for example by a lubricant such as artificial tear fluid. Due to the low frictional forces, it is possible for the user, e.g. the attending physician, to rotate or co-rotate the guide body 62 by using the light guide 71 as a manipulator, i.e. moving it according to a conical or cone-like circular motion. During this movement, the free end F of the light guide 71 is guided by the guide body 62, specifically along a predetermined path, which in the present example corresponds to a circular path or a circular path segment. The free end F of the light guide 71 may thus be positioned and guided in a defined manner over the eye 73 by the guide body 62, and the eye 73 may be exposed to light L, in particular laser light L, by the guidance of the guide body 62 at defined points or along defined segments of the path, for example depending on the treatment requirements, for example for irradiation or exposure of the ciliary muscle for the treatment of glaucoma.

In order to prevent the light guide 71 from being inserted too far into the channel and to prevent the light guide 71 or the free end F from touching the eye 73 during movement along the path, the light guide 71 may have a locking device, for example in the form of a cuff, and/or the channel 63 may have a taper, a stop and the like at the first end E1. It is also possible that the channel 63 is closed at the first end E1, for example in the form of a blind hole, whereby a corresponding closure or base of the channel 63 is transparent for the light L, so that the light L may be applied to the eye 73 essentially unattenuated, for example.

It can also be seen from the illustration according to FIG. 15 that the guide body 62 and the support body 59 may be latched together, wherein, for latching in accordance with the example shown, the guide body 62 has a latching bead 74 which is formed in the first conical surface 68, is of annular circumferential design, and projects from first conical surface 68. Instead of a ring bead 74, latching nubs and the like may also be provided.

In the assembled state, the latching bead 74 engages in a complementary latching groove 75. The latching groove 75 is formed in the second conical surface 69.

The latching groove 75 and the latching bead 74 are dimensioned and designed such that the guide body 62 latches onto the support body 59 and is at least secured against falling out in the direction of the second side 66. Preferably, the latching elements 74, 75 are arranged such that they can at least also absorb torques which may be caused by rotation of the guide body 59 by the light guide 71 as manipulator and/or during movement of the support body 59 by the grip element 61. In particular, this may ensure reliable guidance of the free end F of the light guide 71. In particular, it may be avoided that the guide body 62 moves in the direction of the central axis M or at an angle relative to the support body 59 during handling, or it may be avoided that the guide body 62 tilts relative to the support body during manipulation.

The latching groove 75 and the latching bead 74 are further configured such that the guide body 62 may be latched or inserted into the support body 59 in a non-destructive manner. Furthermore, the latching groove 75 and the latching bead 74 are designed in such a way, in particular with regard to surface properties, that they also form sliding surfaces and enable comparatively easy rotation of the guide body 62.

FIG. 16 shows the device of FIG. 12 with dimensions. Therein, LG denotes a length of the grip element 61, H denotes a height of the support body 59 measured parallel to the central axis M, DU denotes a diameter of the support body 59 and/or the ring bead 65 and/or the bearing edge 70 on the first side 60, and DO denotes a diameter of the support body 59 on the second side 66. Exemplary values, which may be varied in particular within the ranges mentioned further above, are: LG: 69.56 mm, H: 6.90 mm, DU: 18.93 mm, DO: 29.93 mm.

FIG. 17 shows a perspective view of the support body 59 with grip element 61 according to the device of FIG. 12. In particular, FIG. 17 shows the annular or circular latching groove 75 formed in the second conical surface 69, or slide bearing surface, approximately halfway up the support body 59.

FIG. 18 shows a view of FIG. 17 from below, from which in conjunction with FIG. 17 it can be seen in particular that the support body 59 is rotationally symmetrical. Furthermore, it can be seen from FIG. 17 and FIG. 18 that the grip element 61 extends obliquely upwards and is coupled to an outer side of the support body 59, for example is integrally formed therewith or is otherwise fastened.

In a combined view of FIGS. 17, 18 and FIG. 12, it can further be seen that the user may freely select the two channels 63 depending on the angle of rotation of the light guide 71, for example each of the channels 63 may be used for a rotation angle of about 180 degrees, for example. However, it is also possible that one channel 63 may be used for a rotation of the guide body 62 by any angle, in particular between 0 degrees and 360 degrees (and multiples). If the angle of rotation of the guide body 62 is to be limited for specific applications, it may be provided that corresponding stop elements are provided to limit the angle of rotation.

FIG. 19 shows a side view of the guide body 62 of the device according to FIG. 12, showing in particular that the detent bead 74 is formed in the first conical surface 68, i.e. slide bearing surface, protrudes therefrom, is formed approximately halfway up the guide body 62, and is substantially annularly rotationally symmetrical. Exemplary dimensions for the guide body 62 are also indicated in FIG. 19, where DU′ denotes the diameter of the guide body 62 at the first side 60 and DO′ denotes the diameter of the guide body at the second side 66: DU′: 18.75 mm, DO′: 27.59 mm, wherein the values for the diameters may be varied in particular within the ranges mentioned further above.

FIG. 20 shows a view of the support body 59 and guide body 62 of the device 58 according to FIG. 12 from below, i.e. a view looking towards the first side 60. In particular, exit regions 76 of the channels 63 for the light L are evident from FIG. 20.

FIG. 21 shows a top view of the guide body 62, i.e. a view looking towards the second side 66. In FIG. 21, the reference signs 77 designate channel openings of the channels 63 formed on the second side 66 or facing the second side 66, which are formed for insertion, in particular insertion of the light guide 71. In the example shown, the channel openings 77 are provided symmetrically, in particular point-symmetrically to the central axis M, whereby these may be transferred into one another by a 180 degree rotation given that the guide body 62 is freely rotatable about the central axis M.

In embodiments, however, it is also possible for the channels 63, channel openings 77 and outlet regions 76 to be arranged differently. For example, the channels may be arranged differently with respect to the distribution in the circumferential direction around the central axis M and/or the channels 63 may be inclined differently with respect to the central axis M, for example to provide different paths for the free end F. The diameter of the channels may be 0.48 mm, for example, whereby this may be varied, in particular within the ranges indicated above, and, in particular, may be adapted to the diameter of the light guide 71.

FIG. 22 shows a sectional view according to A-A of FIG. 21. FIG. 22 shows in particular the rotationally symmetrical arrangement of the channels 63 on the guide body 62. Furthermore, it can be seen in FIG. 22 that the channels 63 have substantially parallel inner walls, and that the channels 63 extend substantially parallel to the first conical surface 68, i.e. slide bearing surface, and thus, in conjunction with FIG. 14, also substantially parallel to the second conical surface 69, i.e. slide bearing surface. In embodiments, however, the channels 63 may also run non-parallel to the conical surfaces 68 and 69.

FIG. 22 also shows the angle of inclination N of the channels 63 to the central axis M. Due to the symmetry, this is the same for both channels. The angle of inclination N may be 40 degrees, for example, whereby the angle of inclination may be varied in particular within the ranges specified above.

FIG. 23 and FIG. 24 show two different perspective views of the guide body 62. From these figures and in conjunction with FIG. 22, it can be seen in particular that the guide body 62 is essentially designed as a rotationally symmetrical annular body, the channels 63 being provided radially opposite one another. Furthermore, it can be seen that the guide body 62 has an inner cone surface 78 facing inwards, i.e. towards the central axis M, which in particular tapers in a funnel shape towards the first side 60. In the shown exemplary embodiment, the inner cone surface 78 is formed substantially parallel to the first conical surface 68. However, the inner cone surface 78 of the guide body 62 may also be oriented or extend differently.

A funnel-shaped structure provides an advantageous opening and viewing angle, which may be beneficial to a user when placing the support body 59 on the eye 73.

As can be seen from FIGS. 14 to 17, an outer cone surface 79 (FIG. 17) of the support body 59 in the shown exemplary embodiment is also conical in shape and tapers towards the first end 60.

The outer cone surface 79 may be formed essentially parallel to the second conical surface 69 and converge in a funnel shape. However, other paths and shapes of the outer cone surface 79 are also possible.

FIG. 25 schematically shows a use case of the device 58 on an eye 73, wherein the grip element 61 is not shown in FIG. 25 for the sake of clarity. In the example shown, the support body 59 with the ring bead 65 or the support edge 70 is placed on the eye 73, specifically on the cornea 50 of the eye 73, so that the aperture 67 is approximately concentric with the pupil bounded by the iris 53.

The size and geometry of the device 58, in particular the channels 63, their angle of inclination N, and the radii of the guide body 62 and support body 59, are so arranged, and the device 58 is so positioned, that the light exit surface 72 of the light guide 71 is located in the direction of and in the region of the ciliary muscle 52 of the eye 73.

When the device 58 is positioned on the eye 73 as shown, the ciliary muscle 52 may be exposed to light 72, for example in the form of laser pulses. This may influence the ciliary muscle so that increased draining of fluid via the trabecular meshwork of the eye 73 (not shown) may be obtained, which in turn may reduce the intraocular pressure. High intraocular pressure is characteristic of glaucoma. However, the device 58 may also be used for other applications.

By rotating the guide body 62 based on the light guide 71 as a manipulator, as described above, the ciliary muscle 52 may be exposed to light L, e.g. in the form of laser pulses, along the path defined by the guide body 62, according to which the free end F of the light guide 71 moves as a result of the rotation of the guide body 62. The device 58 is therefore suitable for the treatment of glaucoma, but also for other applications.

Due to the slide bearing surfaces, the rotation may be effected comparatively easily by the light guide 71 as a manipulator, with the slide bearing surfaces forming an axial slide bearing in the present case.

The device 58 shown in FIG. 25 has an additional, optional component, specifically an eyelid retaining device 80 for engaging and retaining the eyelids 81 of the eye 73 when the support body 59 is placed on the eyeball.

In the example shown, the eyelid holding device 80 is formed, for example integrally formed, on the support body 62. The eyelid retaining device 80 may, for example, be formed in the manner of an eyelid speculum and, as shown schematically in FIG. 25, may have retaining cups or clips 82 that may be brought into engagement with the eyelids 81 to retain them when the support body 59 is in place.

An eyelid holding device 80, 82 may contribute to the fact that an eye treatment may be carried out more easily, because with such an eyelid holding device 80, 82 no additional eyelid speculum is required. Insofar, an eye treatment with light L, in particular laser light, may be performed with the device 58 as the only ophthalmic instrument to be positioned on the eye 73, at least, the number of ophthalmic instruments required may be reduced.

Overall, it can be seen that the ophthalmic device 58, which forms an ophthalmic instrument, may be advantageously used for eye treatment with light L or laser light, for example in a treatment of glaucoma, is easy to handle, and/or is comparatively easy and/or inexpensive to manufacture.

### LIST OF REFERENCE SIGNS

1 ophthalmic mask
2 mask body
3 first page
4 second page
5 eye
6 central area
7 positioning marker
7.1, 7.2 recess
7.3 indentation
8 light guide
9 distal end
10 notch
11 face-sided edge
12 circular ring
13 groove structure
14 groove
15 inner wall
16 marking
17 ophthalmic device
18 laser light source
19 connection line
20 proximal end
21 handle
22 control unit
23 user interface
24 device unit
50 cornea
51 anterior eye chamber
52 ciliary muscle
53 iris
54 trabecular meshwork
55 lens
56 vitreous body
57 retina
58 further ophthalmic device
59 support body
60 first page
61 grip element
62 guide body
63 retaining element or fixing element, channel
64 rotational movements
65 ring bead
66 second page
67 aperture
68 first conical surface
69 second conical surface
70 support edge
71 light guide
72 light-emitting surface
73 eye
74 latching bead
75 latching groove
76 exit area
77 channel opening
78 inner cone surface
79 outer cone surface
80 eyelid holding device
81 eyelid
82 retaining shell or clamp
A1 to A3 treatment areas
B width
D circle diameter
D1 diameter (through hole)
D2 diameter (distal end)
K convergence angle
L, L′, L″ laser light
U direction of rotation
E1 first end
E2 second end
F free end
LG length grip element
M central axis
DU diameter of support body on the first side
DO diameter of support body on the second side
H height of the support body
LG length grip element
DU′ diameter of guide body on the first side
DO′ diameter of guide body on the second side
N tilt angle

1-22. (canceled)

23. An ophthalmic mask for application to an eyeball of an eye during a punctual irradiation of the eye with light, in particular laser light (L, L′, L″), through a light guide, in particular for use in a treatment or therapy of glaucoma, comprising:
a) a shell-like mask body with a first side for application or support on the eyeball, and a second side facing away from the first side,
b) a plurality of positioning markers for positioning a light guide in the positioning marker, wherein the positioning markers are visible at least on the second side;
c) wherein the shell-like mask body is opaque, at least in a two-dimensional central area, for a light used to treat the eye, in particular laser light (L, L′, L″),
d) an area size of the central area is adapted to an area size of the pupil and iris of an eye to be treated, and
e) the positioning markers are arranged outside the central area, wherein
f) the positioning markers on the mask body are formed as indentations on the face-sided edge and/or as recesses extending from the second side into and/or through the mask body, and the mask body is transparent to the light (L) in the region of the indentations and/or recesses, and wherein
g) a dimension (D1) of the indentations and/or recesses is adapted to a dimension (D2) of a light guide provided for the punctual application of light (L), wherein the dimension of the indentations and/or recesses is designed in terms of shape and/or size such that a distal end of the light guide designed to emit the light (L, L′, L″) is positioned in the indentations and/or recesses, and wherein, in the use case, the distal end of the light guide can be positioned in one of the recesses or indentations for punctual irradiation of the eye with the light (L, L′, L″), so that the light (L, L′, L″) can be irradiated into a treatment area in the eye defined by the indentation or recess.

24. The ophthalmic mask according to claim 23, wherein:
at least one, preferably several, groups of recesses are provided, and centers of at least one, preferably each, group of recesses are each arranged on a path, in particular a circular path, running around the central area; and
a minimum diameter (D) of the path, in particular a diameter of a circular path, is preferably in the range from 9.5 mm to 13 mm, in particular from 10 mm to 12 mm, and/or wherein the mask has at least a two-layer or two-shell structure.

25. The ophthalmic mask according to claim 23, comprising:
a groove structure provided on the second side of the shell body;
wherein:
in each case two recesses or one recess and one indentation are connected by a groove extending therebetween; and
the groove is set up as a guide aid for the light guide.

26. The ophthalmic mask according to claim 23, wherein:
a side wall of one or more indentations and/or recesses has, at least in sections, a conical taper from the second side towards the first side, wherein a minimum diameter of a recess or indentation preferably corresponds essentially to the diameter of the light guide; and
a convergence angle (K) of the conical taper is preferably larger on a side of the recess facing the central area than on a side of the recess facing away from the central area.

27. An ophthalmic device for the treatment, in particular laser treatment, of an eye, comprising:
at least one ophthalmic mask designed to rest on an eyeball of the eye according to claim 23,
a light applicator, in particular laser applicator, with a light guide which has a free distal end, whose shape is adapted to the shape of the positioning markers and can be selectively positioned directly at or in the positioning markers, so that after positioning the distal end directly at or in one of the positioning markers, the eye can be selectively exposed locally to light, in particular laser light (L, L′, L″), through the distal end of the light guide, and
optionally further comprising:
a light source, in particular a laser light source, preferably an Nd:YAG laser source, further preferably a pulsed laser source, which is or can be connected to the light applicator via a connection line;
wherein light (L, L′, L″) which can be generated by the light source can be coupled into the light guide via the connection line for emission at the distal end.

28. An ophthalmic device for the treatment, in particular laser treatment, of an eye, comprising:
a support body with a first side for direct support on an eyeball, in particular for direct support on the cornea of an eye, the support body having a grip element which is set up to hold the support body on the eyeball and/or to press it against the eyeball; and
a guide body which has at least one retaining element or fixing element for releasably holding a free end (F) of a light guide on the guide body in such a way that, when the support body is placed on the eyeball, light (L) can be selectively applied to the eyeball or components thereof through the light guide;
wherein the guide body is movably, in particular slidably, mounted on the support body in such a way that the retaining element or fixing element moves along a predetermined path when the guide body moves relative to the support body.

29. The ophthalmic device according to claim 0, wherein:
the support body has a ring bead on the first side for resting on the eyeball, and/or
the support body has a slide bearing surface and the guide body has a complementary slide bearing surface, which form a slide bearing, preferably an axial slide bearing; and
the slide bearing is further preferably designed as a tapered slide bearing.

30. The ophthalmic device according to claim 28, wherein:
at least one retaining element or fixing element, preferably all retaining elements or fixing elements, is designed as a channel into which the free end (F) of the light guide can be inserted, and
the channel is preferably designed and arranged such that light (L) is applicable to the eyeball or components thereof through the inserted free end (F) of the light guide, upon movement of the guide body, in particular at least locally along the predetermined path.

31. The ophthalmic device according to claim 28, wherein:
the support body comprises a first cone or cone surface forming a slide bearing surface for a complementary second cone or cone surface formed on the guide body; and
the second cone or cone surface rests on the first cone or cone surface.

32. The ophthalmic device according to claim 28, wherein the support body and/or the guide body are/is substantially in the form of a ring body or disk body.

33. The ophthalmic device according to claim 31, wherein the first and/or second cone or disk surface are/is coaxially aligned with the ring body or disk body axis of the respective ring body or disk body.

34. The ophthalmic device according to claim 31, wherein the first cone or cone surface tapers towards the first side and/or wherein the first cone or cone surface is oriented towards the central axis (M) of the support body.

35. The ophthalmic device according to claim 28, wherein:
the retaining element or fixing element is arranged such that the free end (F) of the light guide is inclined at a predetermined angle (N) to the central axis (M) of the guide body;
the angle (N) is preferably between 35 and 45 degrees, in particular at about 40 degrees; and
further preferably, a longitudinal axis of the channel is inclined at the predetermined angle to the central axis of the guide body.

36. The ophthalmic device according to claim 28, wherein:
the guide body is coupled loss-proof to the support body, preferably via complementary coupling elements, such as snap-in surfaces, complementary tongue and groove connections and/or a tongue and groove connection, which are further preferably formed in bearing surfaces, in particular slide bearing surfaces,
the guide body is mounted movably on the support body in such a way that a movement of the guide body and an accompanying movement of the retaining element or fixing element relative to the support body can be effected by a light guide held on or by the retaining element or fixing element as a manipulator, and/or
the guide body is mounted on the support body in such a way that the free end (F) of the light guide held on or by the retaining element or fixing element and facing the first side follows a circular path, which preferably has a diameter in the range between 15 and 25, preferably between 17 mm and 19 mm, in particular approximately 18.75 mm, when the guide body is moved relative to the support body.

37. The ophthalmic device according to claim 28, further comprising:
a device for engaging and retaining the eyelids of the eye when the support body is placed on the eyeball;
wherein the device is preferably formed on the support body.

38. A method comprising one of:
(i) the steps of:
applying an ophthalmic mask according to claim 23 to an eyeball of an eye, and
positioning of a distal end of a light guide of a light applicator, which is designed to emit laser light (L, L′, L″), in one of the positioning markers of the ophthalmic mask, so that the laser light (L, L′, L″) is applicable in a local treatment area (A1, A2, A3) of the eye defined by the positioning marker;
or, in the alternative:
(ii) the steps of:
applying a support body of an ophthalmic device to an eyeball of an eye, and
inserting a distal end of a light guide of a light applicator, which is designed to emit laser light (L, L′, L″), into a retaining element or fixing element of the at least one retaining element or fixing element of the ophthalmic device;
wherein the distal end is detachably held by the retaining element or fixing element on the guide body of the ophthalmic device, and when the support body is placed on the eyeball, and the laser light (L, L′, L″) is locally applicable to the eyeball or components thereof in a targeted manner through the light guide.
